(19) United States
(12) Patent Application Publication
(10) Pub. No.: US 2026/0078084 A1
(43) Pub. Date: Mar. 19, 2026

(54) Composite quaternary ammonium salt cationic collector and its preparation method, and uses thereof

(71) Applicant: ZHENGZHOU NON-FERROUS METALS RESEARCH INSTITUTE CO.LTD OF CHALCO, Zhengzhou (CN)

(72) Inventors: Huanhuan SU, Zhengzhou (CN); Zeshuang KANG, Zhengzhou (CN); Kun YAN, Zhengzhou (CN); Huaxia LI, Zhengzhou (CN); Zhongkai LIU, Zhengzhou (CN); Qiuyun HU, Zhengzhou (CN); Junwei MA, Zhengzhou (CN); Ruixue CAO, Zhengzhou (CN); Yanli ZHANG, Zhengzhou (CN); Tengfei ZHANG, Zhengzhou (CN); Ye TIAN, Zhengzhou (CN); Zekun FAN, Zhengzhou (CN)

(73) Assignee: ZHENGZHOU NON-FERROUS METALS RESEARCH INSTITUTE CO.LTD OF CHALCO, Zhengzhou, HE (CN)

(21) Appl. No.: 19/403,228

(22) Filed: Nov. 27, 2025

Related U.S. Application Data
(63) Continuation of application No. PCT/CN2024/099873, filed on Jun. 18, 2024.

(30) Foreign Application Priority Data
Jun. 19, 2023 (CN) 202310730899.1

(51) Int. Cl. C07C 259/00; C07C 231/12

(52) U.S. Cl. CPC C07C 259/00; C07C 231/12

(57) ABSTRACT

A composite quaternary ammonium salt cationic collector and its preparation method and uses involve: adding a halogenated alkyl group into an alkyl tertiary amine containing amido group to carry out a quaternization reaction to obtain an intermediate; and adding a catalyst into the intermediate to carry out a catalytic reaction to obtain a first mixture; adding an alkyl benzylamine into the first mixture to carry out an amidation reaction to obtain a second mixture; and desalting the second mixture to obtain a composite quaternary ammonium salt cationic collector; a molar ratio of the alkyl tertiary amine, the halogenated alkyl group, and the alkyl benzylamine is (1-1.4):(0.8-1.2):(1-1.4).

### CROSS-REFERENCE TO RELATED APPLICATION

This is a continuation of International Application No. PCT/CN2024/099873, filed on Jun. 18, 2024, which claims priority to Chinese Patent Application No. 202310730899.1, filed on Jun. 19, 2023. The disclosures of the above-referenced applications are hereby incorporated by reference in their entireties.

### TECHNICAL FIELD

The disclosure relates to the technical field of preparation of mineral processing reagent, and in particular to a composite quaternary ammonium salt cationic collector and its preparation method, and uses thereof.

### BACKGROUND

At present, types of bauxite in China are mainly kaolinite and illite type of bauxite. and among collectors for a desilication by reverse flotation of bauxite, amine cationic collectors are most commonly used. Commonly used amine cationic collectors mainly include compounds such as dodecylamine, dodecyl trimethyl quaternary ammonium salt, and dodecyl dimethyl benzyl quaternary ammonium salt. Among the commonly used amine cationic collectors, quaternary ammonium salt collectors are more extensively studied, and quaternary ammonium compounds are mainly alkyl quaternary ammonium salts. Among the alkyl quaternary ammonium salts, a dodecyl trimethyl ammonium chloride has a better effect on a desilication by reverse flotation of bauxite than the dodecylamine. Studies have shown that an action of the dodecyl trimethyl ammonium chloride on aluminosilicate minerals is mainly an electrostatic effect.

At present, applications of cationic collectors in a flotation of potash ore and lepidolite ore or in desilication and impurity removal by reverse flotation of iron ore and bauxite each have their own characteristics, but still have certain shortcomings which are mainly manifested in that: the cationic collectors have a poor biodegradability easily resulting in environmental pollution; the cationic collectors are sensitive to ore slime and have poor selectivity; and the cationic collectors have an efficiency of desilication by reverse flotation which is much lower than an efficiency of direct flotation desilication. In the related art, the cationic collectors mainly include dodecylamine or other long-chain fatty amines. Although polyamines, polyetheramines, tertiary amines, quaternary ammonium salts, and alkylguanidines have been developed after years of research and have greatly promoted a development of cationic collectors, development thereof in a process of desilication by reverse flotation is still relatively slow. In addition, collectors in the related art in the process of desilication by reverse flotation have disadvantages such as inconvenient configuration of reagent, high froths viscosity and poor selectivity.

At present, research on the cationic collectors is mainly focused on two aspects of etheramines and quaternary ammonium salts. However, the two aspects still have disadvantages such as difficult synthesis and high cost, which seriously hinder a promotion and use of a process of desilication by reverse flotation of bauxite. Therefore, how to provide a simple and efficient synthesis method of the cationic collectors to improve a process of the cationic desilication by reverse flotation is a technical problem that needs to be solved urgently.

### SUMMARY

By utilizing one or more embodiments of the disclosure, the technical problem of difficult synthesis and high cost of cationic collectors for desilication by reverse flotation in the related art is solved.

A first aspect of the disclosure provides a method for preparing a composite quaternary ammonium salt cationic collector. The method comprises: adding a halogenated alkyl group into an alkyl tertiary amine containing amido group to carry out a quaternization reaction to obtain an intermediate; and adding a catalyst into the intermediate to carry out a catalytic reaction to obtain a first mixture; adding an alkyl benzylamine into the first mixture to carry out an amidation reaction to obtain a second mixture; and desalting the second mixture to obtain a composite quaternary ammonium salt cationic collector. A molar ratio of the alkyl tertiary amine, the halogenated alkyl group, and the alkyl benzylamine is (1-1.4):(0.8-1.2):(1-1.4).

A second aspect provides use of a method for preparing a composite quaternary ammonium salt cationic collector. The use comprises using the composite quaternary ammonium salt cationic collector obtained by the preparation method of the first aspect in a process of a desilication by reverse flotation of bauxite.

### BRIEF DESCRIPTION OF DRAWINGS

The drawings are incorporated in and constitute a portion of this specification, illustrate embodiments consistent with the disclosure and together with the description, serve to explain the principles of the disclosure.

In order to more clearly illustrate the technical solutions in the embodiments of the disclosure or related art, the following briefly introduces the drawings required for use in the embodiments or related technical descriptions. Obviously, for those skilled in the art, other drawings can be obtained based on these drawings without any creative work.

FIG. 1 shows a schematic flow diagram of a method for preparing a composite quaternary ammonium salt cationic collector according to some embodiments of the disclosure.

FIG. 2 shows a detailed schematic flow diagram of a method for preparing a composite quaternary ammonium salt cationic collector according to some embodiments of the disclosure.

FIG. 3 shows a schematic principle diagram of a method for preparing a composite quaternary ammonium salt cationic collector according to some embodiments of the disclosure.

FIG. 4 shows a schematic flow diagram of a composite quaternary ammonium salt cationic collector used in a desilication by reverse flotation of pure minerals according to some embodiments of the disclosure.

FIG. 5 shows a schematic diagram of an open-circuit process of a composite quaternary ammonium salt cationic collector used in a desilication by reverse flotation of bauxite according to some embodiments of the disclosure.

FIG. 6 shows a schematic diagram of a closed-circuit process of a composite quaternary ammonium salt cationic collector used in a desilication by reverse flotation of bauxite according to some embodiments of the disclosure.

### DETAILED DESCRIPTION

In order to make the purpose, technical solutions and advantages of the embodiments of the disclosure clearer, the technical solutions in the embodiments of the disclosure will be clearly and completely described below in combination with the drawings in the embodiments of the disclosure. Obviously, the described embodiments are only a portion of the embodiments of the disclosure, not all of the embodiments. Based on the embodiments of the disclosure, all other embodiments obtained by those skilled in the art without creative work shall fall within the scope sought by the disclosure.

Unless otherwise specified, various raw materials, reagents, instruments and apparatuses used in the disclosure can be purchased from the market or prepared by existing methods.

At present, main application directions of cationic collectors are as follows.
(1) CN102259062A discloses a method for preparing organosilicon quaternary ammonium compounds, and use of the method in a flotation separation of potassium chloride and sodium chloride and a desilication by reverse flotation and impurity removal of iron ore and bauxite.
(2) CN101337204 discloses use of diquaternary ammonium compounds as flotation collectors in a desilication by reverse flotation of bauxite and iron ore.
(3) CN107442287A discloses use of a Gemini surfactant in a desilication by reverse flotation and impurity removal of potash ore, lepidolite ore or iron ore, and bauxite.
(4) CN1507954A discloses a collector for desilication by reverse flotation and preparation method thereof. The collector is a composite quaternary ammonium salt cationic surfactant containing a quaternary ammonium group and/or a quaternary ammonium salt compound containing benzyl group, and is suitable for a separation by reverse flotation of silicate minerals from iron ores such as magnetite, hematite, and ilmenite, and from ores containing silicate minerals such as bauxite.
(5) CN106238215A discloses a quaternary ammonium salt cationic collector and synthesis method thereof. The diquaternary ammonium salt is synthesized by a two-step method and is mainly used for a desilication by reverse flotation of collophosphate.
(6) CN112657681A discloses a cationic collector and a preparation method and use thereof. The cationic collector is used for desilication of iron ore; desilication of magnesite; desilication of phosphate rock; feldspar flotation, adaptive flotation or silicate flotation.
(7) CN112474061A discloses a preparation and use of a quaternary ammonium salt cationic collector containing an ester group for a reverse flotation of phosphate rock. The quaternary ammonium salt cationic collector contains an ester group, which makes the collector have a good degradation effect. The quaternary ammonium salt cationic collector containing an ester group contains a long carbon chain and a quaternary ammonium group. However, an amount of the quaternary ammonium salt cationic collector containing an ester group in reverse flotation of phosphate rock is too large and a cost is high. In addition, a raw material N, N-dimethylethanolamine is a controlled product and is flammable and toxic.
(8) CN107716116A discloses a preparation method and use of a collector for reverse flotation of iron ore.
(9) CN110605183A discloses a collector for silica and alumina removal by reverse flotation of phosphate rock, and preparation method and use thereof. The collector includes: mixed amines, mixed alcohols and coconut fatty acid methyl ester. CN110038728A discloses a method for reverse flotation of hematite using a highly degradable amine collector. The collector used is a lauramidopropyl dimethylamine oxide. CN109847944A discloses a method for preparing N-(2-hydroxy-1,1-dimethylethyl)alkylamine, suitable for a field of reverse flotation of iron ore.
(10) CN103769307B discloses a water-soluble collector containing a tertiary amine with two hydroxypropyl groups for iron ore flotation. This collector has good water solubility but weak collecting ability and is not easy to form froths.
(11) CN107350084A discloses a triquaternary ammonium salt compound for mineral flotation, which has three mineralophilic groups and is used for a flotation of minerals such as phosphate ore and iron ore.
(12) CN113751207A discloses a collector, and a preparation method and use thereof. A prepared hydrophobic nanoparticle collector is used for a desulfurization and collection by a reverse flotation of fine-grained high-sulfur bauxite, and belongs to a desulfurization collector.
(13) CN113769896A discloses a collector, and a preparation method and use thereof. A prepared collector mainly collects aluminum minerals in a bauxite and belongs to a direct flotation desilication collector, which is different from the collector for desilication by reverse flotation of the disclosure.

The applications of collectors of the above-mentioned related art in a flotation of potash ore and lepidolite ore or in the desilication and impurity removal by reverse flotation of iron ore and bauxite each have their own characteristics, but have certain shortcomings which are mainly manifested in that: the collectors have a poor biodegradability easily resulting in environmental pollution; the collectors are sensitive to ore slime and have poor selectivity; and the collectors have an efficiency of desilication by reverse flotation which is much lower than an efficiency of direct flotation desilication. Moreover, in the related art, the cationic collectors mainly include dodecylamine or other long-chain fatty amines. Although polyamines, polyetheramines, tertiary amines, quaternary ammonium salts and alkylguanidines have been developed after years of research and has greatly promoted a development of cationic collectors, development thereof in a process of the desilication by reverse flotation is still relatively slow. In addition, collectors in the related art in the process of desilication by reverse flotation have disadvantages such as inconvenient configuration of reagent, high froths viscosity and poor selectivity.

At present, a research on the cationic collectors is mainly focused on two aspects of etheramines and quaternary ammonium salts. However, the two aspects still have disadvantages such as difficult synthesis and high cost, which seriously hinder a promotion and application of a process of desilication by reverse flotation of bauxite. Therefore, how to provide a simple and efficient synthesis method of the cationic collectors to improve a process of the cationic desilication by reverse flotation is a technical problem that needs to be solved urgently.

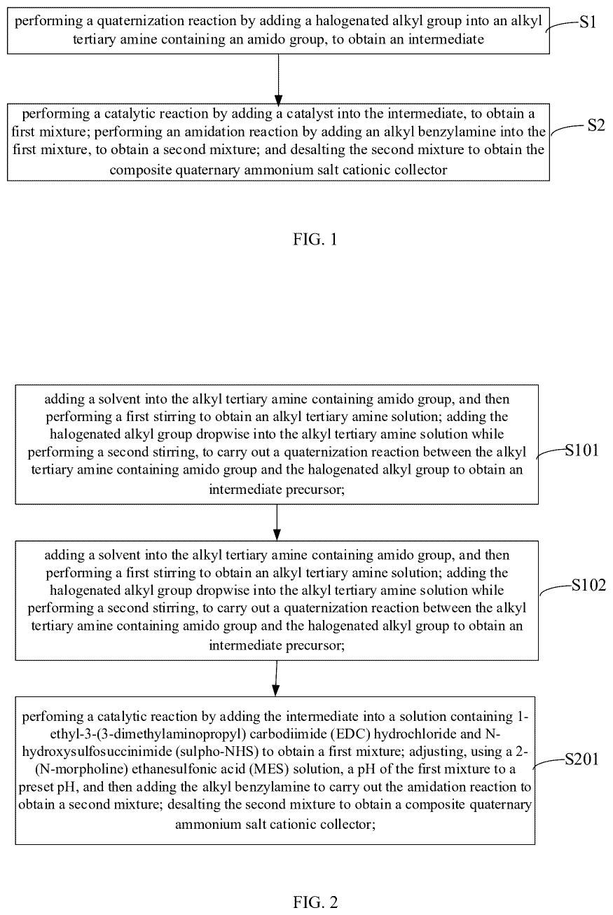

As shown in FIG. 1, a method for preparing a composite quaternary ammonium salt cationic collector according to some embodiments of the disclosure includes:
S1, performing a quaternization reaction by adding a halogenated alkyl group into an alkyl tertiary amine containing an amido group, to obtain an intermediate containing one amide group;
S2, performing a catalytic reaction by adding a catalyst into the intermediate, to obtain a first mixture; performing an amidation reaction by adding an alkyl benzylamine into the first mixture, to obtain a second mixture; and desalting the second mixture to obtain the composite quaternary ammonium salt cationic collector;
a molar ratio of the alkyl tertiary amine, the halogenated alkyl group, and the alkyl benzylamine is (1-1.4):(0.8-1.2):(1-1.4).

In some embodiments of the disclosure, as the molar ratio of the alkyl tertiary amine, the halogenated alkyl group, and the alkyl benzylamine is controlled to be (1-1.4):(0.8-1.2):(1-1.4), the quaternization reaction can be caused to be fully completed, thereby allowing the intermediate to contain a quaternary ammonium group and a carboxylic acid group, and at the same time, forming an amide group in the subsequent amidation reaction, thereby obtaining a composite quaternary ammonium salt cationic collector containing a quaternary ammonium group, an amide group, a straight-chain alkyl group and a benzyl group.

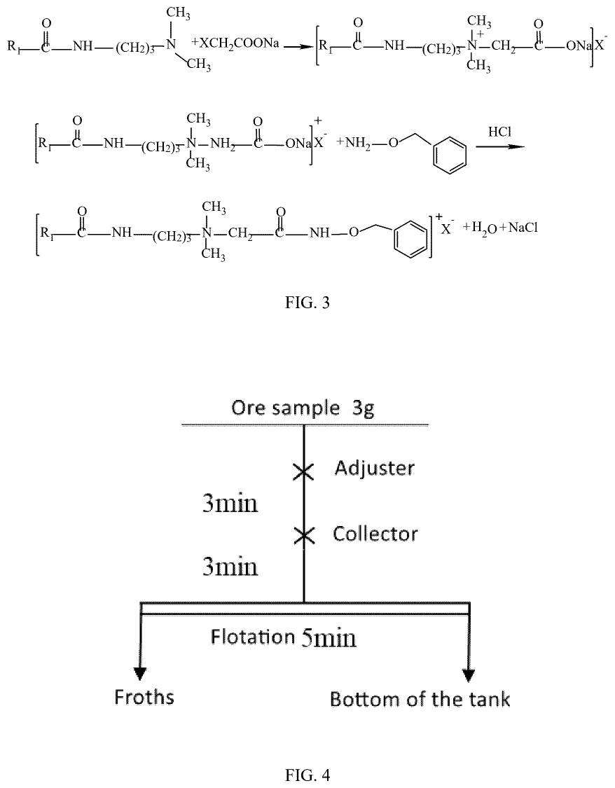

Principles of some optional preparation methods of the composite quaternary ammonium salt cationic collector in the disclosure are shown in FIG. 3, where the R₁ group represents a straight-chain alkane with a hydrocarbon chain length of 11 to 17, and X is Cl or Br.

In some embodiments of the disclosure, the alkyl tertiary amine is an alkyl tertiary amine with a carbon chain length ranging from 12 to 18.

In some embodiments of the disclosure, a carbon chain length of the alkyl tertiary amine is controlled as 12 to 18, so that a long-chain alkane group can be present in the composite quaternary ammonium salt cationic collector. Since the long-chain alkane group is hydrophobic, a hydrophobicity of the composite quaternary ammonium salt cationic collector can be improved, thereby facilitating a smooth progress of collecting process.

In some embodiments of the disclosure, the alkyl tertiary amine includes a lauramide propyl dimethyl tertiary amine and/or a stearamide propyl dimethyl tertiary amine; and/or

the halogenated alkyl group includes a sodium chloroacetate and/or a sodium bromoacetate; and/or the alkyl benzylamine includes a benzyloxyamine hydrochloride.

In some embodiments of the disclosure, it is controlled that the alkyl tertiary amine includes a lauramide propyl dimethyl tertiary amine and/or a stearamide propyl dimethyl tertiary amine; and/or the halogenated alkyl group includes a sodium chloroacetate and/or a sodium bromoacetate; and/or the alkyl benzylamine includes a benzyloxyamine hydrochloride, thereby making it possible to simply synthesize a composite quaternary ammonium salt cationic collector containing a quaternary ammonium group, an amide group, a straight-chain alkane group and a benzyl group.

In some embodiments of the disclosure, a temperature of the quaternization reaction ranges from 30° C. to 55° C., and a time of the quaternization reaction ranges from 18 h to 24 h.

In some embodiments of the disclosure, it is controlled that a temperature of the quaternization reaction ranges from 30° C. to 55° C., and a time of the quaternization reaction ranges from 18 h to 24 h, so that a reaction between the alkyl tertiary amine and the halogenated alkyl group can be fully carried out to obtain an intermediate containing a quaternary ammonium group and a carboxylic acid group.

In some embodiments of the disclosure, a temperature of the amidation reaction ranges from 3° C. to 10° C., and a time of the amidation reaction ranges from 22 h to 26 h.

In some embodiments of the disclosure, it is controlled that a temperature of the amidation reaction ranges from 3° C. to 10° C., and a time of the amidation reaction ranges from 22 h to 26 h, so that an amidation reaction between an intermediate which was subjected to a catalytic reaction and the alkyl benzylamine can be fully carried out to form an amide group, thereby obtaining a composite quaternary ammonium salt cationic collector containing a quaternary ammonium group, an amide group, a straight-chain alkane group and a benzyl group.

In some embodiments of the disclosure, performing a quaternization reaction by adding a halogenated alkyl group into an alkyl tertiary amine containing an amido group to obtain an intermediate, comprises the following sub-steps:
S101, adding a solvent into the alkyl tertiary amine containing amido group, and then performing a first stirring to obtain an alkyl tertiary amine solution; adding the halogenated alkyl group dropwise into the alkyl tertiary amine solution while performing a second stirring, to carry out a quaternization reaction between the alkyl tertiary amine containing amido group and the halogenated alkyl group to obtain an intermediate precursor;
S102, adjusting a pH of the intermediate precursor; condensing and extracting the intermediate precursor which was subjected to an adjustment of the pH, and then evaporating and drying with a desiccant and purifying by vacuum distillation the intermediate precursor to obtain an intermediate containing a quaternary ammonium group and a carboxylic acid group.

In some embodiments of the disclosure, an alkyl tertiary amine solution can be formed by a first stirring and mixing the alkyl tertiary amine with a solvent; and then the halogenated alkyl group is added into the alkyl tertiary amine solution while a second stirring is performed, so that a sufficient quaternization reaction occurs between the halogenated alkyl group and the alkyl tertiary amine containing amido group, thereby obtaining an intermediate precursor containing a quaternary ammonium group and a carboxylic acid group. The intermediate precursor is then subjected to subsequent pH adjustment, condensation, and extraction, and then an evaporation drying and purifying by vacuum distillation can be performed on the intermediate precursor to obtain an intermediate containing a quaternary ammonium group and a carboxylic acid group, thereby further improving a purity of the intermediate.

In some embodiments of the disclosure, the solvent may be an anhydrous ethanol.

In some embodiments of the disclosure, the desiccant may be a sodium sulfate.

In some embodiments of the disclosure, a reagent used for the extraction may be an acetone.

In some embodiments of the disclosure, a time of the first stirring ranges from 10 min to 20 min; and/or a temperature of the second stirring ranges from 35° C. to 45° C., and a time of the second stirring ranges from 19 h to 22 h.

In some embodiments of the disclosure, a time of the first stirring is controlled to range from 10 min to 20 min, so that the alkyl tertiary amine can be fully dispersed in a solvent through the first stirring; a temperature of the second stirring is controlled to range from 35° C. to 45° C. and a time of the second stirring is controlled to range from 19 h˜22h, so that the quaternization reaction can occur between the halogenated alkyl group and the alkyl tertiary amine and the quaternization reaction can be carried out completely through the second stirring, thereby obtaining sufficient intermediates.

In some embodiments of the disclosure, the performing a catalytic reaction by adding a catalyst into the intermediate to obtain a first mixture; performing an amidation reaction by adding an alkyl benzylamine into the first mixture obtain a second mixture; and desalting the second mixture to obtain the composite quaternary ammonium salt cationic collector, comprises the following sub-steps:

S201, preforming a catalytic reaction by adding the intermediate into a solution containing 1-ethyl-3-(3-dimethylaminopropyl) carbodiimide (EDC) hydrochloride and N-hydroxysulfosuccinimide (sulpho-NHS) to obtain a first mixture; adjusting, using a 2-(N-morpholine) ethanesulfonic acid (MES) solution, a pH of the first mixture to a preset pH, and then adding the alkyl benzylamine to carry out the amidation reaction to obtain a second mixture; desalting the second mixture to obtain a composite quaternary ammonium salt cationic collector; where, the preset pH ranges from 5.0-5.5.

In some embodiments of the disclosure, a semi-stable NHS or sulfo-NHS ester can be formed by a catalytic reaction between an EDC hydrochloride, a sulfo-NHS and the intermediate; and then a pH of solution for the catalytic reaction of the EDC hydrochloride, the sulfo-NHS and the intermediate is controlled as a preset pH of 5.0 to 5.5 by a MES solution, so that the semi-stable NHS or sulfo-NHS ester in the first mixture can undergo an amidation reaction with the subsequent alkyl benzylamine, and an amide group can be formed on the intermediate containing a quaternary ammonium group and a carboxylic acid group while a benzyl group can be introduced, thereby obtaining a composite quaternary ammonium salt cationic collector containing a quaternary ammonium group, an amide group, a straight-chain alkane group and a benzyl group on the basis of the intermediate containing a quaternary ammonium group and a carboxylic acid group.

In some embodiments, a structural formula of the composite quaternary ammonium salt cationic collector is as shown in Formula 1:

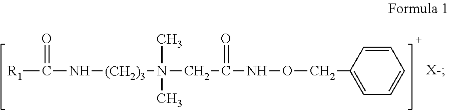

where R₁ group includes a straight-chain alkane group; X includes Cl or Br;
a carbon chain length of the straight-chain alkane group ranges from 11 to 17.

In some embodiments of the disclosure, a structural formula of the composite quaternary ammonium salt cationic collector is controlled to be as shown in Formula 1, and thus a position and distribution way of the quaternary ammonium group, amide group, straight-chain alkane group and benzyl group contained in the composite quaternary ammonium salt cationic collector can be clearly defined, thereby ensuring that the composite quaternary ammonium salt cationic collector has a good water solubility, and also has characteristics of high efficiency of desilication by reverse flotation and environmental friendliness. At the same time, with halide ions introduced as anions into the composite quaternary ammonium salt cationic collector, hydrophobic group of benzyl group in the composite quaternary ammonium salt cationic collector can be better matched, to improve a frothing performance of the composite quaternary ammonium salt cationic collector, to make a froths easy to dissipate after a desilication by reverse flotation and to have a good froths fluidity.

With a length of the straight-chain alkane group in the composite quaternary ammonium salt cationic collector being controlled to be 11 to 17, the R₁ group can be a longer straight-chain alkane group, thereby improving a hydrophobicity of the composite quaternary ammonium salt cationic collector and improving an effectiveness of desilication by reverse flotation of the composite quaternary ammonium salt cationic collector in desilication by reverse flotation of silicate minerals.

Based on a general inventive concept, use of a method for preparing a composite quaternary ammonium salt cationic collector according to some embodiments of the disclosure is provided, which includes using the composite quaternary ammonium salt cationic collector obtained by the preparation method in a process of a desilication by reverse flotation of bauxite. In some embodiments of the disclosure, it is, from the use of the composite quaternary ammonium salt cationic collector in a desilication by reverse flotation of bauxite, found that a pH value suitable of desilication by reverse flotation for the composite quaternary ammonium salt cationic collector in the desilication by reverse flotation ranges from 5-6. A selected bauxite has an aluminum-silicon ratio of 3.55, and a grade of SiO₂ of 14.29%; an aluminum concentrate obtained after the desilication by reverse flotation has an aluminum-silicon ratio of 4.76, and a grade of SiO₂ is reduced as 11.03; the grade of SiO₂ in a tailing is 23.12%, and a recovery rate of SiO₂ thereof is more than 48.31%. A selected bauxite has an aluminum-silicon ratio of 5.69, and a grade of SiO₂ is 10.92%; an aluminum concentrate obtained after the desilication by reverse flotation has a yield of 77.41%, an aluminum-silicon ratio of the aluminum concentrate is 7.23, and a grade of SiO₂ is reduced as 8.97%; and recovery rate % of Al₂O₃ reaches 80.19%.

The use is realized based on the above-mentioned preparation method. Steps of the preparation method can refer to the above-mentioned embodiments. The use, since adopting a portion or all of the technical solutions of the above-mentioned embodiments, has at least all the advantageous effects brought by the technical solutions of the above-mentioned embodiments, which will not be described one by one here.

Based on a general inventive concept, a composite quaternary ammonium salt cationic collector is provided according to some embodiments of the disclosure which is prepared through the method for preparing the composite quaternary ammonium salt cationic collector.

The composite quaternary ammonium salt cationic collector is realized based on the preparation method of the above-mentioned composite quaternary ammonium salt cationic collector. Steps of the method for preparing the composite quaternary ammonium salt cationic collector can refer to the above-mentioned embodiment. The composite quaternary ammonium salt cationic collector, since adopting a portion or all of the technical solutions of the above-mentioned embodiment, has at least all the advantageous effects brought by the technical solutions of the above-mentioned embodiments, which will not be described one by one here.

Based on a general inventive concept, a use of the composite quaternary ammonium salt cationic collector is provided according to some embodiments of the disclosure. The composite quaternary ammonium salt cationic collector is used in a process of desilication by reverse flotation of bauxite.

The use of the composite quaternary ammonium salt cationic collector in process of desilication by reverse flotation of bauxite is realized based on the above-mentioned composite quaternary ammonium salt cationic collector. Steps of the method for preparing the composite quaternary ammonium salt cationic collector can refer to the above-mentioned embodiments. The use of the composite quaternary ammonium salt cationic collector in the process of the desilication by reverse flotation of bauxite, since adopting a portion or all of the technical solutions of the above-mentioned embodiments, has at least all the advantageous effects brought by the technical solutions of the above-mentioned embodiments, which will not be described one by one here.

The disclosure is further illustrated below with reference to the following examples. It should be understood that these examples are only used to illustrate the disclosure but are not used to limit the scope of the disclosure. The experimental methods in the following examples where the conditions are not specified are generally determined in accordance with national standards in China. If there is no corresponding national standard in China, it is proceed according to the general international standards, conventional conditions, or the conditions recommended by the manufacturer.

### Example 1

As shown in FIG. 2, a method for preparing composite quaternary ammonium salt cationic collector includes the following steps.
(1) molar ratios of various raw materials are controlled to be n (alkyl tertiary amine): n (halogenated alkyl group): n (benzyloxyamine hydrochloride)=1.2:1:1.2.
(2) a lauramide propyl dimethyl tertiary amine is added into a reactor, and an anhydrous ethanol is added and stirred for 10 min to 20 min; and then a sodium chloroacetate is added dropwise for 1 h, and then stirred at 40° C. for a reaction for 20 h; after the reaction is over, 1 mol/L HCl is added for acidification to a pH 5; a vacuum condensing and an extracting with acetone (2×20 mL) are performed, and a combining of organic layers is performed; a washing with salt water solution (3×20 mL), a drying with sodium sulfate and an evaporating are performed; and a residue is purified by vacuum distillation to obtain a first product.
(3) the first product is added into a solution containing EDC and sulfo-NHS, and a pH value of the solution is adjusted as 5.5 with MES as a buffer; and then a benzyloxyamine hydrochloride solution is added dropwise; a reaction at 4° C. for 24 h is performed after an addition of the benzyloxyamine hydrochloride solution is completed; and it is subjected to a desalting to obtain a composite quaternary ammonium salt cationic collector, which is recorded as a collector A.

### Example 2

Comparing Example 2 with Example 1, a difference between Example 2 and Example 1 is as follows.

A method for preparing composite quaternary ammonium salt cationic collector, includes the following steps.
(1) molar ratios of various raw materials are controlled to be n (alkyl tertiary amine): n (halogenated alkyl group): n (benzyloxyamine hydrochloride)=1:1.2:1.
(2) a lauramide propyl dimethyl tertiary amine is added into a reactor, and an anhydrous ethanol is added and stirred for 10 min to 20 min; and then a sodium chloroacetate is added dropwise for 1 h, and then stirred at 35° C. for a reaction for 19 h; after the reaction is over, 1 mol/L HCl is added for acidification to pH 5; a vacuum condensing and an extracting with acetone (2×20 mL) are performed, and a combining of organic layers is performed; a washing with salt water solution (3×20 mL), a drying with sodium sulfate and an evaporating are performed; and a residue is purified by vacuum distillation to obtain a first product.
(3) the first product is added into a solution containing EDC and sulfo-NHS, and a pH value of the solution is adjusted as 5.5 with MES as a buffer; and then a benzyloxyamine hydrochloride solution is added dropwise, a reaction at 4° C. for 23 h is performed after an addition of the benzyloxyamine hydrochloride solution is completed; and it is subjected to a desalting to obtain a composite quaternary ammonium salt cationic collector, which is recorded as a collector B.

### Example 3

Comparing Example 3 with Example 1, a difference between Example 3 and Example 1 is as follows.

A method for preparing composite quaternary ammonium salt cationic collector, includes the following steps.
(1) molar ratios of various raw materials are controlled to be n (alkyl tertiary amine): n (halogenated alkyl group): n (benzyloxyamine hydrochloride)=1.2:0.8:1.4.
(2) a lauramide propyl dimethyl tertiary amine is added into a reactor, and an anhydrous ethanol is added and stirred for 10 min to 20 min; and then a sodium chloroacetate is added dropwise for 1 h, and then stirred at 45° C. for a reaction for 19 h; after the reaction is over, 1 mol/L HCl is added to acidify a pH to be 5; a vacuum condensing and an extracting with acetone (2×20 mL) are performed, and a combining of organic layers is performed; a washing with salt water solution (3×20 mL), a drying with sodium sulfate and an evaporating are performed; and a residue is purified by vacuum distillation to obtain a first product.
(3) the first product is added into a solution containing EDC and sulfo-NHS, and a pH value of the solution is adjusted as 5.5 with MES as a buffer; and then a benzyloxyamine hydrochloride solution is added dropwise, a reaction at 4° C. for 25 h is performed after an addition of the benzyloxyamine hydrochloride solution is completed; and it is subjected to a desalting to obtain a composite quaternary ammonium salt cationic collector, which is recorded as a collector C.

Data of related experiments and effectiveness are as follows.
1. Collectors A, B, C and conventional collectors for desilication by reverse flotation (such as dodecyl trimethyl ammonium chloride, lauramide) are used in a process of desilication by reverse flotation of kaolinite pure mineral.

An experiment of desilication by reverse flotation is carried out on an XFG hanging trough flotation machine with a main shaft speed of 1650 r/min. During the experiment, 3 g of mineral is weighed and placed in a flotation tank of 40 mL. A certain amount of distilled water (25 mL˜30 mL) is added, followed by stirring and slurry conditioning for 1 min; then an adjuster is added to act for 3 min; then a collector is added, followed by stirring for 3 min; and a desilication by reverse flotation is performed for 5 min. During a process of desilication by reverse flotation, a froths product is collected, and the froths product and a product in tank are dried and weighed separately, and a recovery rate is calculated. A flow chart of experiment is shown in FIG. 4. Results of experiment are shown in Table 1.

TABLE 1
Results of Comparative experiments of collectors for
desilication by reverse flotation of kaolinite

| | Recovery rates of different collectors for desilication | | | | | |
|---|---|---|---|---|---|---|
| | by reverse flotation of kaolinite (%) | | | | | |
| | | dodecyl | | | | |
| Concentration | | trimethyl | | | | |
| of collector | | ammonium | | Collector | Collector | Collector |
| (×10⁻² mol/L) | Lauramide | chloride | Dodecylamine | A | B | C |
| 0.05 | 63.95 | 59.96 | 46.83 | 71.93 | 69.50 | 70.84 |
| 0.1 | 69.51 | 79.81 | 67.19 | 80.86 | 78.65 | 79.99 |
| 0.25 | 73.53 | 83.99 | 82.46 | 91.30 | 87.99 | 88.86 |
| 0.35 | 81.66 | 87.1 | 85.55 | 99.27 | 95.85 | 97.34 |
| 0.4 | 86.53 | 93.46 | 87.71 | 99.88 | 96.55 | 98.79 |

It can be seen from Table 1 that among the prepared collectors, Collector A has a stronger ability to collect kaolinite and surpasses the conventional collector for desilication by reverse flotation in the related art.
2. Collectors A, B, C and conventional collectors for desilication by reverse flotation in related art are used for a desilication by reverse flotation of illite pure mineral.

Steps of experiment are the same as above, and results of experiment are shown in Table 2.

TABLE 2
Results of Comparative experiments of collectors
for desilication by reverse flotation of illite

| | Recovery rates of different collectors for desilication | | | | | |
|---|---|---|---|---|---|---|
| | by reverse flotation of illite (%) | | | | | |
| | | dodecyl | | | | |
| Concentration | | trimethyl | | | | |
| of collector | | ammonium | | Collector | Collector | Collector |
| (×10⁻² mol/L) | Lauramide | chloride | Dodecylamine | A | B | C |
| 0.05 | 58.00 | 61 | 56.18 | 72.03 | 69.57 | 71.54 |
| 0.1 | 66.38 | 65 | 58.25 | 84.64 | 79.66 | 81.61 |
| 0.25 | 73.61 | 67 | 65.13 | 94.03 | 90.74 | 92.13 |
| 0.35 | 76.14 | 76 | 83.54 | 97.54 | 95.49 | 96.67 |
| 0.4 | 77.00 | 82 | 84.00 | 98.12 | 96.32 | 97.37 |

It can be seen from Table 2 that among the three prepared reagents, collector A has a stronger ability to collect illite and surpasses the conventional collector for desilication by reverse flotation in the related art.
3. Collectors A, B, C and conventional collectors for desilication by reverse flotation in related art are used for a desilication by reverse flotation of pyrophyllite pure minerals.

Steps of experiment are the same as above, and results of experiment are shown in Table 3.

TABLE 3
Results of Comparative experiments of collectors for
desilication by reverse flotation of pyrophyllite

| | Recovery rates of different collectors for desilication | | | | | |
|---|---|---|---|---|---|---|
| | by reverse flotation of pyrophyllite (%) | | | | | |
| | | dodecyl | | | | |
| Concentration | | trimethyl | | | | |
| of collector | | ammonium | | Collector | Collector | Collector |
| (×10⁻² mol/L) | Lauramide | chloride | Dodecylamine | A | B | C |
| 0.05 | 61.07 | 69.56 | 62.65 | 89.65 | 87.66 | 88.53 |
| 0.1 | 75.66 | 79.57 | 79.58 | 95.84 | 92.91 | 93.86 |
| 0.25 | 82.74 | 85.13 | 82.46 | 98.36 | 95.12 | 96.54 |
| 0.35 | 83.33 | 89.85 | 85.31 | 99.04 | 96.34 | 97.89 |
| 0.4 | 88.94 | 94.52 | 88.11 | 99.98 | 97.33 | 98.32 |

The experiment results in Table 3 show that Collector A has a stronger ability to collect pyrophyllite and surpasses the conventional collector for desilication by reverse flotation in the related art.
4. Collectors A, B, C and conventional collectors for desilication by reverse flotation in related art are used for a desilication by reverse flotation of bauxite. The bauxite used is a bauxite of Henan, and has a component analysis shown in Table 4 and Table 5. where, 1227 refers to dodecyl dimethyl benzyl ammonium chloride, and 1221 refers to dodecyl trimethyl ammonium chloride.

Analysis results of chemical composition of experimental bauxites are shown in Table 4 and Table 5:

TABLE 4
Analysis results of chemical composition of bauxite minerals (%)

| | | | | | | | | | Loss on | | |
|---|---|---|---|---|---|---|---|---|---|---|---|
| Ore samples | Al₂O₃ | SiO₂ | Fe₂O₃ | CaO | K₂O | Na₂O | TiO₂ | MgO | Ignition | A/S | S |
| Mines of | 57.22 | 16.37 | 6.46 | 0.38 | 1.31 | 0.06 | 2.91 | 0.29 | 13.72 | 3.50 | 0.59 |
| Henan | | | | | | | | | | | |

TABLE 5
Analysis results of mineral phase composition of bauxite (%)

| Types of | | | | | | | | | | | |
|---|---|---|---|---|---|---|---|---|---|---|---|
| ore samples | diaspore | Kaolinite | Illite | Chlorite | Pyrophyllite | Siderite | Hematite | Pyrite | Calcite | Anatase | Rutile |
| Mines of | 52 | 18 | 12.5 | 4 | 2 | 2 | 2.5 | 1.1 | 0.7 | 2.4 | 0.5 |
| Henan | | | | | | | | | | | |

Steps and process of Experiments are as follows.

Step 1: Slurry conditioning:
250 g of ore sample is put into a flotation tank of 1.5 L of the XFG hanging trough flotation machine; a tap water is added; and a slurry conditioning is performed for 3 minutes at a speed of flotation machine of 2100 r/min.

Step 2: Addition of adjuster:
After Step 1, a hydrochloric acid and an inhibitor are added to adjust a pH to be between 5 and 6, and then the slurry conditioning is performed for 2 minutes.

Step 3: Addition of collector:
After Step 2, a collector is added to perform a desilication by reverse flotation for 5 minutes, and then an air valve is opened to perform a flotation and froth scraping.

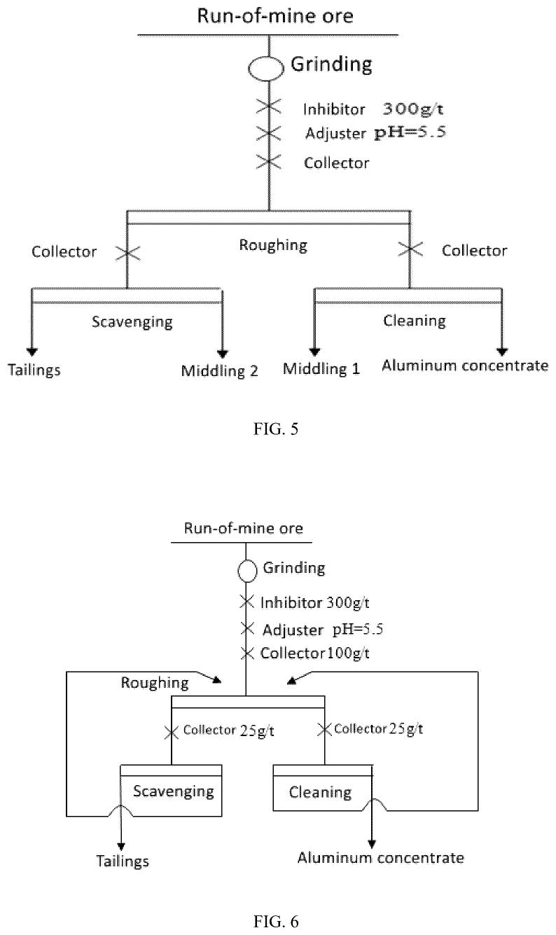

Step 4: Processing of product obtained through desilication by reverse flotation:
After a process of the desilication by reverse flotation is completed, samples (tailings) which is mixed by the froths product of flotation with middling and a product (concentrates) in the tank are respectively dried, weighed, tested, and indicators of the products are calculated. Experiment processes are shown in FIG. 5, where the experiment processes include one roughing, one cleaning, and one scavenging process. The floated froths and middling are mixed as tailings, and products on a bottom of the tank is used as concentrates. Results are shown in Table 6.

TABLE 6
Experiment results of reagents for desilication by reverse flotation

| Reagents | | | | | | Recovery | recovery |
|---|---|---|---|---|---|---|---|
| for | | Yield | AL | Si | | rate of | rate of |
| flotation | Product | (%) | (%) | (%) | A/S | Aluminum (%) | SiO₂ (%) |
| Collector | Concentrates | 70.68 | 58.75 | 14.9 | 3.94 | 72.71 | 63.17 |
| C (300 g/t) | Tailings | 29.32 | 53.16 | 20.94 | 2.54 | 27.29 | 36.83 |
| | Total | 100 | 57.11 | 16.67 | 3.43 | 100.00 | 100.00 |
| Collector | Concentrates | 73.02 | 58.25 | 14.62 | 3.98 | 75.11 | 66.02 |
| B (300 g/t) | Tailings | 26.98 | 52.24 | 20.36 | 2.57 | 24.89 | 33.98 |
| | Total | 100 | 56.63 | 16.17 | 3.50 | 100.00 | 100.00 |
| Collector | Concentrates | 75.82 | 60.09 | 14.29 | 4.21 | 79.55 | 65.53 |
| A (150 g/t) | Tailings | 24.18 | 48.43 | 23.57 | 2.05 | 20.45 | 34.47 |
| | Total | 100 | 57.27 | 16.53 | 3.46 | 100.00 | 100.00 |
| 1231 | Concentrates | 73.24 | 56.6 | 14.78 | 3.83 | 75.71 | 65.92 |
| (300 g/t) | Tailings | 26.76 | 49.69 | 20.09 | 2.47 | 24.29 | 32.74 |
| | Total | 100 | 54.55 | 16.42 | 3.32 | 100 | 100.00 |
| 1227 | Concentrates | 75.54 | 58.63 | 14.34 | 4.09 | 77.7 | 67.45 |
| (350 g/t) | Tailings | 24.46 | 51.99 | 21.38 | 2.43 | 22.3 | 32.56 |
| | Total | 100 | 57.01 | 16.06 | 3.55 | 100 | 100.00 |
| Dodecylamine | Concentrates | 56.73 | 58.12 | 13.85 | 4.20 | 58.9 | 47.29 |
| (150 g/t) | Tailings | 43.27 | 55.00 | 20.24 | 2.72 | 41.1 | 52.71 |
| | Total | 100.00 | 56.77 | 16.61 | 3.42 | 100 | 100.00 |
| N-[3- | Concentrates | 71.53 | 58.73 | 15.42 | 3.81 | 73.3 | 66.69 |
| (Dimethylamino) | Tailings | 28.47 | 53.75 | 19.35 | 2.78 | 26.7 | 33.31 |
| propyl]lauramide | Total | 100 | 57.31 | 16.54 | 3.47 | 100 | 100.00 |
| (50 g/t) | | | | | | | |

It can be seen from Table 6 that among the three prepared reagents: collector A, collector B and collector C, a required dosage of the collector A is less, and at the same time the concentrates have a higher yield, and a higher aluminum-silicon ratio and recovery rate of Aluminum are high. Collector A, compared with conventional collectors in related art, has a less dosage. At the same time, by observing an increase in the yield and aluminum-silicon ratio of various concentrates, it can be known that Collector A has a better effectiveness in desilication by reverse flotation.
5. Collector A and conventional collectors in related art are used in desilication by reverse flotation of Mines of Chongqing.

Analysis results of chemical composition of Mines of Chongqing are shown in Tables 7 and 8. Steps of experiment are the same as Experiment 4, and results of experiment are shown in Table 9.

TABLE 8
Analysis results of mineral phase composition of bauxite (%)

| Types of | | | | | | | | | | |
|---|---|---|---|---|---|---|---|---|---|---|
| ore samples | Diaspore | Boehmite | Kaolinite | Illite | Chlorite | Pyrophyllite | Siderite | Pyrite | Anatase | Rutile |
| Mines of | 40 | 16 | 17.5 | 4.5 | 12 | / | 3 | 3.7 | 2 | 0.5 |
| Chongqing | | | | | | | | | | |

TABLE 9
Analysis results of chemical composition of bauxite minerals (%)

| | | | | | | | | | Loss on | | |
|---|---|---|---|---|---|---|---|---|---|---|---|
| Ore samples | Al₂O₃ | SiO₂ | Fe₂O₃ | CaO | K₂O | Na₂O | TiO₂ | MgO | Ignition | A/S | S |
| Mines of | 58.78 | 13.04 | 9.96 | 0.11 | 0.49 | 0.09 | 2.45 | 0.48 | 12.72 | 4.51 | 2.00 |
| Chongqing | | | | | | | | | | | |

TABLE 9
Experiment results of various collectors at preferred
dosages for desilication by reverse flotation

| Reagents | | | | | | Recovery | recovery |
|---|---|---|---|---|---|---|---|
| for | | Yield | AL | Si | | rate of | rate of |
| flotation | Product | (%) | (%) | (%) | A/S | Aluminum (%) | SiO₂ (%) |
| Dodecylamine | Concentrates | 68.86 | 59.65 | 12.14 | 4.91 | 72.02 | 64.35 |
| (400 g/t) | Tailings | 31.14 | 51.24 | 14.87 | 3.45 | 27.98 | 35.65 |
| | Total | 100 | 57.03 | 12.99 | 4.39 | 100.00 | 100.00 |
| 1231 | Concentrates | 75.29 | 58.99 | 11.76 | 5.02 | 77.16 | 70.72 |
| (250 g/t) | Tailings | 24.71 | 53.19 | 15.81 | 3.36 | 22.84 | 31.20 |
| | Total | 100 | 57.37 | 12.52 | 4.51 | 100.00 | 100.00 |
| 1227 | Concentrates | 77.09 | 60.68 | 11.48 | 5.29 | 80.75 | 71.26 |
| (500 g/t) | Tailings | 22.91 | 48.69 | 15.59 | 3.12 | 19.25 | 28.76 |
| | Total | 100 | 57.93 | 12.42 | 4.66 | 100.00 | 100.00 |
| Collector | Concentrates | 76.61 | 61.41 | 11.17 | 5.50 | 81.70 | 68.49 |
| A (150 g/t) | Tailings | 23.39 | 45.06 | 16.83 | 2.68 | 18.30 | 31.51 |
| | Total | 100 | 57.59 | 12.49 | 4.61 | 100.00 | 100.00 |
| Dodecyl | Concentrates | 79.13 | 60.5 | 12.06 | 5.02 | 83.19 | 75.38 |
| tertiary | Tailings | 20.87 | 46.36 | 14.95 | 3.10 | 16.81 | 24.65 |
| amine | Total | 100 | 57.55 | 12.66 | 4.55 | 100.00 | 100.00 |
| (1100 g/t) | | | | | | | |
| N-Lauryl- | Concentrates | 77.41 | 64.84 | 8.97 | 7.23 | 80.19 | 59.91 |
| 1,3-propane | Tailings | 22.59 | 54.89 | 20.57 | 2.67 | 19.81 | 40.09 |
| diamine | Total | 100.00 | 62.59 | 11.59 | 5.40 | 100.00 | 100.00 |
| (1200 g/t) | | | | | | | |

It can be seen from Table 9 that different collectors have different preferred dosages for desilication by reverse flotation, and a dosage of collector A is relatively less. By observing and comparing the aluminum-silicon ratio and yield of various concentrates, the yield of concentrates obtained after reverse flotation with collector A reaches 76.61%, the aluminum-silicon ratio is high and reaches 5.5, and the recovery rate of aluminum in the concentrates is high and the recovery rate of aluminum in the concentrates reaches 81.7%.
6. An example of use of collector A in open-circuit experiment of desilication by reverse flotation of bauxite of Shanxi.

Analysis results of chemical composition of Mines of Shanxi are shown in Tables 10 and 11. Steps of experiment are the same as Experiment 4, and results of experiment are shown in Table 12.

TABLE 10
Analysis results of chemical composition of bauxite minerals (%)

| | | | | | | | | | Loss on | | |
|---|---|---|---|---|---|---|---|---|---|---|---|
| Ore samples | Al₂O₃ | SiO₂ | Fe₂O₃ | CaO | K₂O | Na₂O | TiO₂ | MgO | Ignition | A/S | S |
| Mines of | 50.69 | 14.29 | 8.86 | 2.27 | 0.63 | 0.05 | 1.99 | 1.02 | 16.57 | 3.55 | 2.65 |
| Shanxi | | | | | | | | | | | |

TABLE 11
Analysis results of mineral phase composition of bauxite (%)

| Ore samples | diaspore | dolomite | Kaolinite | Illite | Pyrophyllite | Siderite | Pyrite | Calcite | Anatase | Rutile | goyazite |
|---|---|---|---|---|---|---|---|---|---|---|---|
| Mines of | 43.6 | 3.3 | 22.13 | 10.87 | / | 8.6 | 4.4 | 2.3 | 1.5 | 0.5 | 0.4 |
| Shanxi | | | | | | | | | | | |

Steps of experiment are the same as Experiment 4, and results of experiment are shown in Table 12.

TABLE 12
Results of Comparative experiments of collectors
in desilication by reverse flotation of bauxite

| | | | | | | recovery | recovery |
|---|---|---|---|---|---|---|---|
| Collector | | Yield | Al₂O₃ | SiO₂ | | rate of | rate of |
| type | Product | (%) | (%) | (%) | A/S | Al₂O₃ (%) | SiO₂ (%) |
| 1227 | Concentrates | 68.62 | 53.50 | 11.18 | 4.79 | 72.16 | 54.65 |
| | Tailings | 31.38 | 45.13 | 20.29 | 2.22 | 27.84 | 45.35 |
| | Total | 100 | 50.87 | 14.04 | 3.62 | 100 | 100 |
| 1231 | Concentrates | 68.66 | 53.13 | 11.86 | 4.48 | 72.46 | 55.86 |
| | Tailings | 31.34 | 44.25 | 20.53 | 2.16 | 27.54 | 44.14 |
| | Total | 100 | 50.35 | 14.58 | 3.45 | 100 | 100 |
| Dodecylamine | Concentrates | 55.32 | 53.06 | 12.08 | 4.39 | 58.27 | 45.88 |
| | Tailings | 44.68 | 47.05 | 17.64 | 2.67 | 41.73 | 54.12 |
| | Total | 100 | 50.37 | 14.56 | 3.46 | 100 | 100 |
| Lauramide | Concentrates | 56.87 | 51.8 | 11.23 | 4.61 | 59.44 | 44.94 |
| | Tailings | 43.13 | 46.6 | 18.14 | 2.57 | 40.56 | 55.06 |
| | Total | 100 | 49.56 | 14.21 | 3.49 | 100 | 100 |
| Collector | Concentrates | 67.06 | 54.25 | 10.72 | 5.06 | 71.51 | 50.95 |
| A | Tailings | 32.94 | 44 | 21.01 | 2.09 | 28.49 | 49.05 |
| | Total | 100 | 50.87 | 14.11 | 3.61 | 100 | 100 |

According to Table 12, a comprehensive comparison about the yield of the concentrates and the aluminum-silicon ratio, and a recovery rate of silicon in the tailings shows that the collectors of some embodiments of the disclosure, compared with conventional collectors in the related art, have better selectivity and stronger collection ability.
7. An example of use of collector A in closed-circuit experiment of desilication by reverse flotation of bauxite.

Steps of experiment are the same as Experiment 4. Process of experiment is shown in FIG. 6. Results of experiment are shown in Table 13:

TABLE 13
Results of Comparative experiments of collectors
in desilication by reverse flotation of bauxite

| | | | | | | recovery | recovery |
|---|---|---|---|---|---|---|---|
| | | Yield | Al₂O₃ | SiO₂ | | rate of | rate of |
| Collector | Product | (%) | (%) | (%) | A/S | Al₂O₃ (%) | SiO₂ (%) |
| Collector | Concentrates | 70.16 | 55 | 10.83 | 5.08 | 74.85 | 51.59 |
| A | Tailings | 30.84 | 42.05 | 23.12 | 1.82 | 25.15 | 48.41 |
| | Total | 100 | 51.56 | 14.73 | 3.50 | 100 | 100 |
| 1231 | Concentrates | 69.76 | 54.23 | 11.55 | 4.70 | 74.40 | 55.07 |
| | Tailings | 30.24 | 43.05 | 21.74 | 1.98 | 25.60 | 44.93 |
| | Total | 100 | 50.85 | 14.63 | 3.48 | 100 | 100 |

Table 13 shows that in the closed-circuit experiment, the collectors according to some embodiments of the disclosure achieved better effectiveness of desilication by reverse flotation.

One or more technical solutions in the embodiments of the disclosure may also have at least the following technical effects or advantages.
(1) According to the method for preparing the composite quaternary ammonium salt cationic collector of some embodiments of the disclosure, a prepared composite quaternary ammonium salt compound is more readily degradable and more hydrophilic due to a presence of an amide group in a molecule thereof, and is therefore more friendly to the environment and humans in terminal applications; and a molecule of the prepared composite quaternary ammonium salt compound has a hydrophobic benzyl group and a quaternary ammonium group that is affinity to groups of mineral surface, and thus a separation by reverse flotation of oxide ores and salt minerals can be enhanced, and a characteristic of high efficiency in desilication by reverse flotation can be achieved.
(2) According to a perpetration method of composite quaternary ammonium salt cationic collector of some embodiments of the disclosure, a prepared composite quaternary ammonium salt cationic collector has a good water solubility, and is environmentally friendly and safe.
(3) According to application of the method for preparing the composite quaternary ammonium salt cationic collector of some embodiments of the disclosure, an obtained composite quaternary ammonium salt cationic collector has a strong collecting ability for silicate minerals such as kaolinite and illite, is suitable for desilication by reverse flotation of bauxite, has good selectivity, and has a low dosage.
(4) According to the application of the method for preparing the composite quaternary ammonium salt cationic collector of some embodiments of the disclosure, the obtained composite quaternary ammonium salt cationic collector is used for flotation in acidic pulp, to be advantageous to a recovery for utilization; and the collector provided by the embodiments of the disclosure, compared with conventional collectors for desilication by reverse flotation, has a small dosage, a high aluminum-silicon ratio of the concentrates, and a high efficiency of desilication by reverse flotation.
(5) According to the use of the method for preparing the composite quaternary ammonium salt cationic collector of some embodiments of the disclosure, a froths is easy to disappear after flotation, and a froths fluidity is good.

The above technical solution provided by the embodiments of the disclosure has the following advantages compared with the related art.

A method for preparing a composite quaternary ammonium salt cationic collector is provided according to an embodiment of the disclosure, an alkyl tertiary amine and a halogenated alkyl group are used to carry out a quaternary ammonium reaction to generate an intermediate having a quaternary ammonium group and a carboxylic acid group; then a catalyst is used to carry out a catalytic reaction on the intermediate; and then an alkyl benzylamine is added to carry out an amidation reaction, so that the carboxylic acid group can react with the alkyl benzylamine under a condition of the catalyst to generate an amide group and simultaneously obtain a benzyl group, thereby obtaining a composite quaternary ammonium salt cationic collector containing a quaternary ammonium group, an amide group, a straight-chain alkane group and a benzyl group. The straight-chain alkane group makes this cationic collector have good hydrophobicity. The quaternary ammonium group can generate an electrostatic interaction by reacting with a surface of siliceous mineral particles to be adsorbed on a surface of mineral particles to float out the siliceous mineral particles. The quaternary ammonium group can perform a desilication by reverse flotation on silicate minerals in acidic pulp. At the same time, a presence of the amide group makes the composite quaternary ammonium salt cationic collector easier to degrade and have more hydrophilic characteristics, and thus the composite quaternary ammonium salt cationic collector is more friendly to environment and human in terminal applications. At the same time, a hydrophobic benzyl group in molecules of the composite quaternary ammonium salt cationic collector can enhance a flotation separation of oxide ores and salt minerals, improve a performance of froths and a desilication selectivity of the composite quaternary ammonium salt cationic collector, and thus the composite quaternary ammonium salt cationic collector has characteristics of high efficiency of desilication by reverse flotation and environmental friendliness. Since an overall synthesis process only requires three steps of reaction and conditions for the reaction are mild, this cationic collector can be synthesized more simply and at a lower cost. Various embodiments of the disclosure may be presented in a form of a range. It should be understood that the description in the form of a range is merely for convenience and brevity and should not be understood as a rigid limitation on the scope of the disclosure. Therefore, it should be considered that the range description has specifically disclosed all possible sub-ranges and single numerical values within the range. For example, description of a range from 1 to 6 should be considered to have specifically disclosed sub-ranges, such as from 1 to 3, from 1 to 4, from 1 to 5, from 2 to 4, from 2 to 5, from 3 to 5, etc., as well as single numbers within that range, such as 1, 2, 3, 4, and 5, and this applies regardless of the range. Additionally, whenever a numerical range is indicated herein, it is indicated to include any cited numeral (fractional or integral) within the indicated range.

In the disclosure, unless otherwise indicated, directional words such as “upper” and “lower” used refer specifically to directions in the drawings. Additionally, in the description of the disclosure, the terms “including”, “comprising” and the like indicate “including but not limited to”. In the disclosure, relational terms such as “first” and “second” are used merely to distinguish one entity or operation from another entity or operation, but do not necessarily require or imply any actual relationship or order between these entities or operations. In the disclosure, “and/or” describes the association relationship of associated objects, indicating that three relationships may exist. For example, A and/or B can indicates: A exists alone, A and B exist at the same time, and B exists alone. A and B can be singular or plural. In the disclosure, “at least one” indicates one or more, and “a plurality” indicates two or more. The expressions “At least one”, “at least one of the following” or similar expressions refer to any combination of these limitations, including any combination of single limitations or plural limitations. For example, “at least one of a, b, or c” or “at least one of a, b, and c” can both indicate: a, b, c, ab (i.e., a and b), ac, bc, or abc, where a, b, c can be single or multiple.

The above description is merely specific embodiments of the disclosure, which enables those skilled in the art to understand or implement the disclosure. Various modifications to these embodiments will be readily apparent to those skilled in the art, and the generic principles defined herein may be implemented in other embodiments without departing from the spirit or scope of the disclosure. Thus, the disclosure will not be limited to the embodiments shown herein but is to be accorded the widest scope consistent with the principles and novel features disclosed herein.

What is claimed is:

1. A method for preparing a composite quaternary ammonium salt cationic collector, comprising:
performing a quaternization reaction by adding a halogenated alkyl group into an alkyl tertiary amine containing amido group to obtain an intermediate; and
performing a catalytic reaction by adding a catalyst into the intermediate to obtain a first mixture, performing an amidation reaction by adding an alkyl benzylamine into the first mixture to obtain a second mixture, and desalting the second mixture to obtain a composite quaternary ammonium salt cationic collector;
wherein, a molar ratio of the alkyl tertiary amine, the halogenated alkyl group, and the alkyl benzylamine is (1-1.4):(0.8-1.2):(1-1.4).

2. The method according to claim 1, wherein the alkyl tertiary amine is an alkyl tertiary amine with a carbon chain length ranging from 12 to 18.

3. The method according to claim 2, wherein the alkyl tertiary amine comprises a lauramide propyl dimethyl tertiary amine and/or a stearamide propyl dimethyl tertiary amine; and/or,
the halogenated alkyl group comprises a sodium chloroacetate and/or a sodium bromoacetate; and/or,
the alkyl benzylamine comprises a benzyloxyamine hydrochloride.

4. The method according to claim 2, wherein a temperature of the quaternization reaction ranges from 30° C. to 55° C., and a time of the quaternization reaction ranges from 18 h to 24 h.

5. The method according to claim 1, wherein a temperature of the amidation reaction ranges from 3° C. to 10° C., and a time of the amidation reaction ranges from 22 h to 26 h.

6. The method according to claim 1, wherein the performing a quaternization reaction by adding a halogenated alkyl group into an alkyl tertiary amine containing amido group to obtain an intermediate, comprises:
adding a solvent into the alkyl tertiary amine containing amido group, and then performing a first stirring to obtain an alkyl tertiary amine solution; adding the halogenated alkyl group dropwise into the alkyl tertiary amine solution while performing a second stirring, to carry out the quaternization reaction between the alkyl tertiary amine containing amido group and the halogenated alkyl group to obtain an intermediate precursor; and
adjusting a pH of the intermediate precursor; condensing and extracting the intermediate precursor which was subjected to an adjustment of the pH, followed by evaporating and drying with a desiccant and purifying by vacuum distillation the intermediate precursor to obtain the intermediate containing a quaternary ammonium group and a carboxylic acid group.

7. The method according to claim 6, wherein a time of the first stirring ranges from 10 min to 20 min; and/or,
a temperature of the second stirring ranges from 35° C. to 45° C., and a time of the second stirring ranges from 19 h to 22 h.

8. The method according to claim 1, wherein the performing a catalytic reaction by adding a catalyst into the intermediate to obtain a first mixture, performing an amidation reaction by adding an alkyl benzylamine into the first mixture to obtain a second mixture, and desalting the second mixture to obtain a composite quaternary ammonium salt cationic collector, comprises:
performing the catalytic reaction by adding the intermediate into a solution containing 1-ethyl-3-(3-dimethylaminopropyl) carbodiimide hydrochloride and N-hydroxysulfosuccinimide to obtain a first mixture; adjusting, with a 2-(N-morpholine) ethanesulfonic acid solution, a pH of the first mixture to a preset pH, and then performing the amidation reaction by adding the alkyl benzylamine to obtain the second mixture; desalting the second mixture to obtain the composite quaternary ammonium salt cationic collector;
wherein, the preset pH ranges from 5.0-5.5.

9. The method according to claim 1, wherein a structural formula of the composite quaternary ammonium salt cationic collector is as shown in Formula 1: 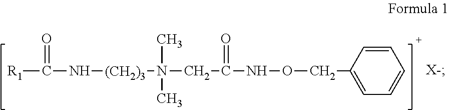
formula 1;
wherein R¹ group comprises a straight-chain alkane group; X comprises Cl or Br;
a carbon chain length of the straight-chain alkane group ranges from 11 to 17.

10. A use of a preparation method of a composite quaternary ammonium salt cationic collector, comprising: using the composite quaternary ammonium salt cationic collector obtained by the method according to claim 1 in a desilication by reverse flotation of bauxite.

11. A composite quaternary ammonium salt cationic collector, prepared by the method for preparing the composite quaternary ammonium salt cationic collector according to claim 1.

12. A use of the composite quaternary ammonium salt cationic collector according to claim 11, wherein the composite quaternary ammonium salt cationic collector is used in a process of a desilication by reverse flotation of bauxite.
